(19) United States
(12) Patent Application Publication
(10) Pub. No.: US 2022/0259680 A1
(43) Pub. Date: Aug. 18, 2022

(54) SARS CoV-2 INFECTIVITY DETERMINATION ASSAY

(71) Applicants: Bio-Rad Laboratories, Inc., Hercules, CA (US); Biodesix, Inc., Boulder, CO (US)

(72) Inventors: Dianna MAAR, Mountain House, CA (US); Monica HERRERA, Dublin, CA (US); George KARLIN-NEUMANN, Palo Alto, CA (US); Josh SHINOFF, Emeryville, CA (US); Audrey AUDETAT, Boulder, CO (US); Scott HUTTON, Boulder, CO (US); Hestia MELLERT, Boulder, CO (US); Leisa JACKSON, Boulder, CO (US); Gary PESTANO, Boulder, CO (US)

(21) Appl. No.: 17/670,811

(22) Filed: Feb. 14, 2022

Related U.S. Application Data
(60) Provisional application No. 63/150,050, filed on Feb. 16, 2021.

(51) Int. Cl. C12Q 1/6888

(52) U.S. Cl. CPC C12Q 1/6888; C12Q 1/6851

(57) ABSTRACT

Methods and compositions for characterizing a biological sample (e.g., comprising an infectious agent) from a subject are provided. Methods can include detecting linkage of nucleic acids that are linked in a viable cell or organism but that become degraded and thus unlinked in inviable cells or organisms and then characterizing the subject based on the quantity of linked and unlinked sequences.

### CROSS-REFERENCE TO RELATED PATENT APPLICATIONS

The present patent application claims benefit of priority to U.S. Provisional Patent Application No. 63/150,050, filed on Feb. 16, 2021, which is incorporated by reference for all purposes.

### BACKGROUND OF THE INVENTION

The SARS-CoV-2 coronavirus causes the coronavirus disease 2019 (COVID-19) and can be dispersed through respiratory droplets and direct human contact. Spread of SARS-CoV-2 can be from individuals with severe, moderate, or mild symptoms as well as individuals without symptoms. Rates of infection are dependent on contagiousness and exposure to infected individuals in the population. While most have only mild or even no symptoms, those who experience severe symptoms can suffer injury to organs including the lungs, heart, and circulatory system.

Three main types of tests are available to help detect individuals with active viral infection. Nucleic acid amplification testing (NAAT), such as those that use Polymerase Chain Reaction (PCR) can detect the virus itself during pre-symptomatic, asymptomatic, and symptomatic infection. Individuals may continue to show low copy numbers of the virus using PCR following convalescence. Direct antigen tests detect viral protein fragments and are most effective for symptomatic infections and is most useful in providing rapid results. In contrast, serology (antibody) tests can identify an individual's immune response to the virus and may indicate prior infection. The analytic performance of these tests and adherence to their validated uses are useful for delivering high quality and reliable results.

Various health organizations have issued recommendations for congregate settings such as hospitals, long-term care facilities, penitentiary systems, factories and in highly technical workspaces and occupations. For those in high-risk environments with limited ability to socially distance, frequent testing is useful to identify active infection and to monitor numbers of previous infection. However, policies for return to schools, work, and other social activities that bring people together, including sports and entertainment are still not clearly defined. Beyond general guidelines for quarantining and testing suspected symptomatic individuals, there is very little consensus on frequency of testing in these situations.

A caveat of testing is the varied sensitivity of the available tests including the nucleic acid tests, that is driven by the general inability of most technologies to accurately quantify viral loads during SARS-CoV-2 infection. Further, tests usually report qualitative outputs including positive, negative, and invalid results only, with some manufacturers' limiting the ability of the users to review or report the underlying quantitative or relatively quantitative values.

### BRIEF SUMMARY OF THE INVENTION

In some embodiments, a method of characterizing an infectious agent in a subject is provided. In some embodiments, the method comprises: providing a first sample from the subject comprising infectious agent nucleic acids; partitioning the first sample into a plurality of first partitions; detecting in the first partitions the presence or absence of a first infectious agent nucleic acid and a second infectious agent nucleic acid, wherein the first infectious agent nucleic acid and the second infectious agent nucleic acid are covalently linked in a viable infectious agent nucleic acid; determining (a) the number of first partitions that contain the first infectious agent nucleic acid linked to the second infectious agent nucleic acid and (b) the number of first partitions that contain the first infectious agent nucleic acid without the second infectious agent nucleic acid or (c) the number of first partitions that contain the second infectious agent nucleic acid without the first infectious agent nucleic acid (for example determining the percentage of linked first and second nucleic acid (# of partitions showing linked signal/(# of partitions showing linked+# of partitions showing unlinked, i.e., one but not both of first infectious agent nucleic acid and second infectious agent nucleic acid); and characterizing the infectious agent in the subject based on the determining of (a) and (b) or (a) and (c).

In some embodiments, the determining comprises determining (b) and (c) and the characterizing is based on the determining of (a) and (b) and (c).

In some embodiments, the characterizing comprises comparing (a), (b), (c) or a combination thereof to one or more threshold value.

In some embodiments, the method further comprises: providing a second sample from the subject comprising infectious agent nucleic acids, wherein the second sample was obtained from the subject at a later time point than the first sample; partitioning the second sample into a plurality of second partitions; detecting in the second partitions the presence or absence of a first infectious agent nucleic acid and a second infectious agent nucleic acid; determining (a′) the number of second partitions that contain the first infectious agent nucleic acid linked to the second infectious agent nucleic acid, (b′) the number of second partitions that contain the first infectious agent nucleic acid without the second infectious agent nucleic acid and (c′) the number of second partitions that contain the second infectious agent nucleic acid without the first infectious agent nucleic acid; wherein the characterizing comprises comparing (a) to (a′), (b) to (b′), (c) to (c′) or a combination thereof. In some embodiments, the second sample was obtained from the subject at least 24 hours (e.g., 1-10, 1-5, 1-3, 1-2 days) after the first sample was obtained.

In some embodiments, the method further comprises detecting in the partitions a control nucleic acid and wherein the determining comprises normalizing: (a) the number of first partitions that contain the infectious agent nucleic acid linked to the second infectious agent nucleic acid, and b) the number of first partitions that contain the first infectious agent nucleic acid without the second infectious agent nucleic acid, and/or (c) the number of first partitions that contain the second infectious agent nucleic acid without the first infectious agent nucleic acid, to the number of partitions containing the control nucleic acid.

In some embodiments, the characterizing comprises categorizing the infectious agent as viable or degraded.

In some embodiments, the infectious agent is a virus. In some embodiments, the infectious agent is a virus selected from the group consisting of SARS-CoV-2, influenza, and respiratory syncytial virus (RSV). In some embodiments, the infectious agent is SARS-CoV-2. In some embodiments, the first infectious agent nucleic acid comprises at least a detectable portion of nucleocapsid (N) gene N1 and the second infectious agent nucleic acid comprises at least a detectable portion of N gene N2.

In some embodiments, the infectious agent is a bacterium or a mycoplasma.

In some embodiments, the first infectious agent nucleic acid and the second infectious agent nucleic acid are separated by 100-200,000 (e.g., 100-10,000) nucleotides from each other in the viable infectious agent nucleic acid.

In some embodiments, the subject is a human.

In some embodiments, the partitions are droplets in an emulsion or microwells or nanowells.

Also provided is a method of characterizing an infectious agent in a subject. In some embodiments, the method comprises: providing a first sample from the subject comprising infectious agent nucleic acids; determining (a) an amount of first infection agent nucleic acid linked to the second infection agent nucleic acid, (b) an amount of first infectious agent nucleic acid unlinked to second infectious agent nucleic acid and (c) optionally an amount of second infectious agent nucleic acid unlinked to first infectious agent nucleic acid; and characterizing the infectious agent in the subject based on the determining of (a), (b) and optionally (c).

### BRIEF DESCRIPTION OF THE DRAWINGS

FIG. 1 depicts a representation for detection of linkage in the SARS-CoV-2 N1 and N2 gene targets.

FIG. 2. depicts a hypothetical representation of SARS-CoV-2 infection in a mild and severe case of Covid-19 and an example of changes of linkage detection as the patients pass through different stages of infection.

FIG. 3a-l. Serial Detection of linked and unlinked viral genomes in one donor over time. Partition plots are shown for one individual over the duration of covid-19 infection. Nasal swab specimens were analyzed prior to molecular positivity, through the pre-symptomatic, asymptomatic, symptomatic, asymptomatic (recovery) and convalescence (molecular negative), using a SARS CoV-2 ddPCR test. Specimens were recorded as positive (greater than or equal to 20 copies of N1 and N2) or negative (fewer than 20 copies of N1 and N2). FIG. 3b shows exemplary labels for clusters representing linked N1, N2 (circles), while the N1 and N2 gene targets are unlinked (rectangles and hexagons), respectively. The change in the linked and unlinked clusters represents the shift from intact viral genomes (circles) to an increase in fragmented genomes (rectangle and hexagon). Clusters that do not contain either N1 or N2 are either empty partitions (diamonds negative) or contain the human control gene RPP30 only (trapezoid RPP30+).

FIG. 4. Normalized copies of N1 and N2 to RPP30 Representing Viral Load in Representative Donor 1. Normalized copies of N1 and N2 to RPP30 were calculated as copies per microliter reactions to determine a serial score for copy numbers using RPP30 control gene as the normalizer for each day of specimen collection. Viral load score=[(N1+N2)/2]/RPP30.

FIG. 5: Key for viewing FIG. 6a-e, which shows the various stages of SARS-CoV-2 infection measured in three donors serially using ddPCR in respiratory specimens.

FIG. 6 a-e. Representative Partition Plots for Multiple Donors Through COVID-19 Infection. Partition plots are shown for individuals over the duration of covid-19 infection. Nasal swab specimens were analyzed prior to molecular positivity (pre-symptoms on day 0), pre-symptomatic (day 2), symptomatic (peak molecular counts on day 5), asymptomatic (recovering) and convalescent (molecular negative), using a SARS CoV-2 ddPCR test. FIG. 6b (donor 1) shows exemplary labels for clusters representing linked N1, N2 (circles), while the N1 and N2 gene targets are unlinked (rectangles and hexagons), respectively. The change in the linked and unlinked clusters represents the shift from intact viral genomes (circles) to an increase in fragmented genomes (rectangle and hexagon). Clusters that do not contain either N1 or N2 are either empty partitions (diamonds) or contain the human control gene RPP30 only (trapezoid).

FIG. 7a-d. Representative gene linked and partially linked partition plots for donors with varying symptoms (a) asymptomatic; (b) mildly symptomatic and (c, d) severe symptoms (required hospitalization and/or oxygen). (a) Donor 4: Asymptomatic for duration of infection; Days 3 and 6 are shown. (b) Donor 2: Mild symptoms; Days 5 and 9 are shown. (c) Donor 5: Severe symptoms; Days 6 and 11 are shown. (d) Donor 6: Severe symptoms, Days 9 and 12 are shown. The kinetics of viral load and linkage are similar in all donors (see also Table 5). FIG. 7a (day 6) shows exemplary labels for clusters representing linked N1, N2 (circles), while the N1 and N2 gene targets are unlinked (rectangles and hexagons), respectively. The change in the linked and unlinked clusters represents the shift from intact viral genomes (circles) to an increase in fragmented genomes (rectangle and hexagon). Clusters that do not contain either N1 or N2 are either empty partitions (diamonds negative) or contain the human control gene RPP30 only (trapezoid RPP30+).

FIG. 7a: Donor 4 was asymptomatic for covid duration. 2D plots show linked (left) and un-linked (right) genomes.

FIG. 7b: Donor 2 showed mild symptoms. 2D plots show linked (left) and un-linked (right) viral genomes.

FIG. 7c: Two donors with severe covid symptoms. Donor 5 (top panel) and donor 6 (bottom panel). 2D partition plots show linked (left) and unlinked (right) patterns associated with the viral genomes.

FIG. 8a, b depict partition plots for convalescent donors at multiple timepoints following SARS-CoV-2 infection. Partition plots are shown for individuals over the duration of covid-19 infection. Nasal swab specimens were analyzed prior to molecular positivity (pre-symptoms on day 0), pre-symptomatic (day 2), symptomatic (peak molecular counts on day 5), asymptomatic (recovering) and convalescent (molecular negative), using a SARS CoV-2 ddPCR test. FIG. 6b (donor 1) shows exemplary labels for clusters representing linked N1, N2 (circles), while the N1 and N2 gene targets are unlinked (rectangles and hexagons), respectively. The change in the linked and unlinked clusters represents the shift from intact viral genomes (circles) to an increase in fragmented genomes (rectangle and hexagon). Clusters that do not contain either N1 or N2 are either empty partitions (diamonds) or contain the human control gene RPP30 only (trapezoid).

FIG. 9 depicts linkage examples in COVID-19 clinical specimens. 2D plots show similar mean percent linkage scores and clustering in individuals who are either asymptomatic or symptomatic for COVID-19. Figures are labels to show exemplary labels for clusters representing linked and unlinked N1 and N2 gene target. Clusters that do not contain either N1 or N2 are either empty partitions (negative) or contain the human control gene RPP30 only (RPP30+).

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, analytical chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document.

The term “amplification reaction” refers to any in vitro method for multiplying the copies of a target sequence of nucleic acid in a linear or exponential manner. Such methods include, but are not limited to, polymerase chain reaction (PCR).

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing or linear amplification.

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990. Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

A “primer” refers to a polynucleotide sequence that hybridizes to a sequence on a target nucleic acid and optionally serves as a point of initiation of nucleic acid synthesis. Primers can be of a variety of lengths. In some embodiments, a primer is less than 100 or 50 nucleotides in length, e.g., from about 10 to about 900, from about 15 to about 80, or from about 30-85 to about 30 nucleotides in length. The length and sequences of primers for use in an amplification reaction (e.g., PCR) can be designed based on principles known to those of skill in the art; see, e.g., PCR Protocols: A Guide to Methods and Applications, Innis et al., eds, 1990. The primer can include or be completely formed from DNA, RNA or non-natural nucleotides. In some embodiments, a primer comprises one or more modified and/or non-natural nucleotide bases. In some embodiments, a primer comprises a label (e.g., a detectable label).

A nucleic acid, or portion thereof, “hybridizes” to another nucleic acid under conditions such that non-specific hybridization is minimal at a defined temperature in a physiological buffer. In some cases, a nucleic acid, or portion thereof, hybridizes to a conserved sequence shared among a group of target nucleic acids. In some cases, a primer, or portion thereof, can hybridize to a primer binding site if there are at least about 6, 8, 10, 12, 14, 16, or 18 contiguous complementary nucleotides, including “universal” nucleotides that are complementary to more than one nucleotide partner. Alternatively, a primer, or portion thereof, can hybridize to a primer binding site if there are fewer than 1 or 2 complementarity mismatches over at least about 12, 14, 16, or 18 contiguous complementary nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 60, 65, 70, 75, or 80° C.

As used herein, “nucleic acid” refers to DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

As used herein, the term “partitioning” or “partitioned” refers to separating a sample into a plurality of portions (e.g., compartments), or “partitions.” Partitions can be solid or fluid. In some embodiments, a partition is a solid partition, e.g., a microchannel a nanowell or a well (i.e., in a multi-well microtiter dish). In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil).

### DETAILED DESCRIPTION OF THE INVENTION

The inventors have discovered that linkage between different sequences in a cell or organism can be used to assess viability of the cell or organism. For example, in a cell or organism in which two sequences are linked in its genome, detection and quantification of linkage of the two sequences can be correlated to the cell's or organism's viability and status, allowing one to characterize (e.g., categorize) the cell or organism based on the linkage observed.

As one example, the inventors have discovered that quantifying linkage of linked sequences in a virus, SARS CoV-2, can be an indicator of the viruses' viability and thus whether a person carrying the virus is likely to be contagious or not. For example, following initial infection, the number of linked sequences in SARS-CoV-2 increase greatly during the initial days of infection, but at some point the number of linked sequences peak while the number of unlinked sequences (i.e., where one sequence but not the other is measured in a partition), increase. Thus, by assessing the number of linked sequences as well as the number of unlinked sequences, one can categorize and thus characterize the virus obtained from a subject, for example categorizing the virus as being infectious and thus the subject being more or less contagious.

As discussed in more detail below, digital amplification methods, such as for example droplet digital PCR (ddPCR), can be used to measure linkage. For example, by partitioning a sample into many partitions, one can separate individual nucleic acid molecules in different partitions. If the two sequences are covalently linked, for example being on the same nucleic acid, then partitions should include both linked sequences (or more if more than two sequences are detected). In contrast, if the two sequences are no longer linked, for example due to degradation of the cell's or organism's nucleic acid, then the proportion of partitions having one or the other but not both sequences will increase. Detection and quantification of degradation of the cell's or organism's nucleic acid allows one to categorize the cell or organism in a sample as being viable or under duress or otherwise inviable.

Any disease or genetic condition can be assessed with the methods described herein where the nucleic acid targets are linked in one condition and separated due to degradation in another condition. In some embodiments, linked nucleic acid sequences occur in an infectious organism (i.e., an infectious agent) and measurement of linkage can be used to assess the viability of the organism in the host. For example, the relative contagiousness of a subject carrying the organism, or the effect of a treatment can be assessed based on quantification of linkage.

Exemplary infectious organisms include, but are not limited to viruses, bacteria, fungi and mycoplasma. Exemplary viruses include but are not limited to RNA viruses or DNA viruses, e.g., Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including but not limited to SARS-CoV-2), Influenza virus A, Influenza virus B, Measles virus, Polyomavirus, Human Papilloma virus, Respiratory syncytial virus (RSV), Adenovirus, Coxsackie virus, Dengue virus, Mumps virus, Poliovirus, Rabies virus, Rous sarcoma virus, Reovirus, Yellow fever virus, Ebola virus, Marburg virus, Zika virus, Lassa fever virus, Eastern Equine Encephalitis virus, Japanese Encephalitis virus, St. Louis Encephalitis virus, Murray Valley fever virus, West Nile virus, Lymphocytic choriomeningitis virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, Human Immunodeficiency virus type-2; echovirus; parvovirus; vaccinia virus; molluscum virus; JC virus; and arboviral encephalitis virus. Further viruses that can be analyzed for linkage are described in, e.g., U.S. Pat. No. 9,944,998. Any linked sequences, i.e., sequences that are linked during at least a part of the life cycle of the virus can be used to monitor linkage as described herein.

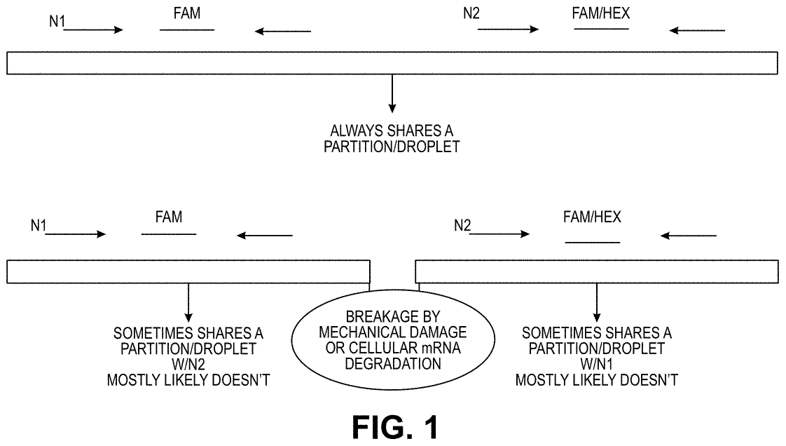

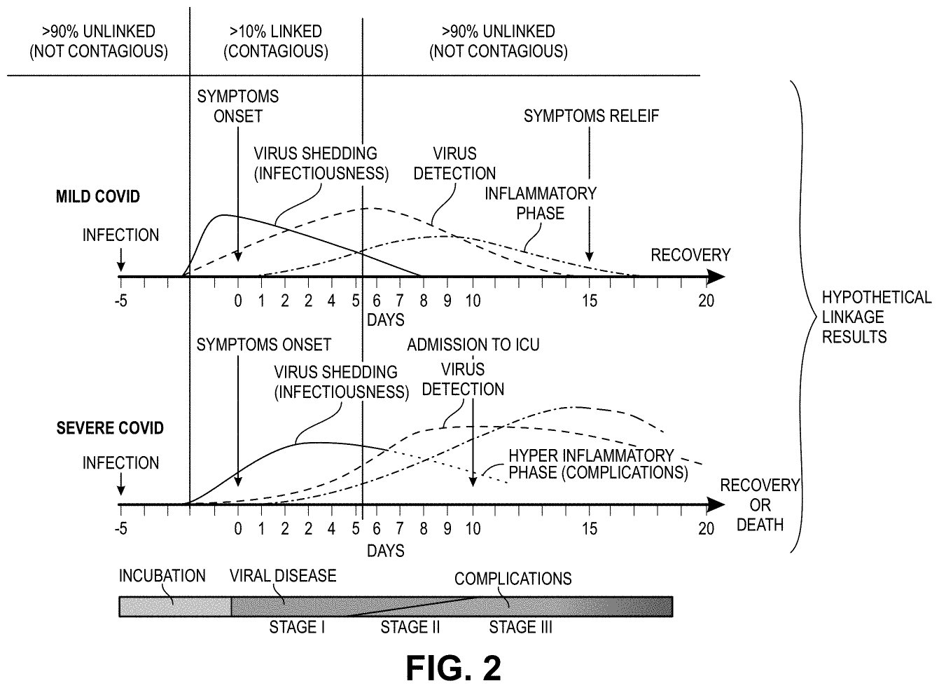

In some embodiments, the linked sequences are from the SARS-CoV-2 genome. A variety of SARS-CoV-2 nucleotide sequences are available, including those described in Wang et al, J Clin Microbiol Infect Dis. 2020 Apr. 24: 1-7 and in NCBI SARS-CoV-2 Resources. As shown in the examples, detection of linkage between the N1 and N2 sequences of the nucleocapsid (N) coding sequence can be used (see, e.g., FIG. 1), however, other linked sequences in the SARS-CoV-2 genome can also be used. For example, the two linked sequences can be from for example the coding sequence of another SARS CoV-2 protein, e.g., spike (S), membrane (M), open reading frame (ORF), or envelope (E) proteins. In some embodiments a first sequence is detected from a first coding sequence and a second sequence is detected from a second coding sequence, wherein the two coding sequences are on the same nucleic acid of the viable virus's genome.

In some embodiments, the infectious organism is a bacteria. Exemplary bacteria can include but are not limited to Escherichia coli, Salmonella, Helicobacter pylori, Neisseria gonorrhoeae, Neisseria meningitides, Staphylococcus and Streptococcal bacteria.

The distance between the two linked target sequences can be any length that allows for monitoring viability of the detected organism at the specificity and sensitivity desired. In some embodiments, the two target nucleic acid sequences are separated, when linked, by 10-10,000 nucleotides, e.g., 50-5,000 nucleotides, 100-1000 nucleotides, e.g., at least 10, 50, 100, 500, or 1000 nucleotides but in some embodiments, no more than 200,000, 100,000, 50,000, 25,000, 10,000, 5,000, 2,000 or 1,000 nucleotides. As noted above, in some embodiments, the linkage of more than two (e.g., 3, 4, or more) nucleic acid sequences are detected by the methods described herein. The distances indicated above can also be applied between the second and third, or third and fourth, etc., target nucleic acid sequences in the linked genome of the organism.

The sample from which linkage is detected can be any biological sample. In the case of an infectious organism, the sample can be a subject known to have (e.g., having received a clinical test indicative of infection) or suspected of being exposed or infected by the infectious organism. Biological samples can be obtained from any biological organism, e.g., an animal, plant, fungus, pathogen (e.g., bacteria or virus), or any other organism. In some embodiments, the biological sample is from an animal, e.g., a mammal (e.g., a human or a non-human primate, a cow, horse, pig, sheep, cat, dog, mouse, or rat), a bird (e.g., chicken), or a fish. A biological sample can be any tissue or bodily fluid obtained from the biological organism, e.g., blood, a blood fraction, or a blood product (e.g., serum, plasma, platelets, red blood cells, and the like), sputum, saliva or bronchoalveolar lavage (BAL), tissue (e.g., kidney, lung, liver, heart, brain, nervous tissue, thyroid, eye, skeletal muscle, cartilage, or bone tissue); cultured cells, e.g., primary cultures, explants, and transformed cells, stem cells, stool, urine, etc. In some embodiments, the sample is a sample comprising cells. The test specimen could also be in containers existing outside of the host and be detected for example in wastewater or other effluent, or as aerosolized droplets generated by air exchange systems, or on the surface of objects, walls, floors, etc.

In some embodiments, the sample is contacted with one or more preservatives until it is partitioned and linkage is detected. Alternatively, the sample need not be contacted with a preservative. Especially when multiple samples are obtained and compared, so long as each sample is stored in substantially the same way, a comparison of the frequency of linkage occurrence of the nucleic acids can be made between samples regardless of the presence or absence of preservatives. In general, the sample is not exposed to nucleases or other reagents that cleave the nucleic acids prior to partitioning and detection, and may, in fact, comprise the entire intact organism itself (e.g. a virion).

Methods of detecting nucleic acid linkage using partitioning and droplet (or other partitioning) digital amplification have been described. See, e.g., U.S. Patent Application No. 2012/0322058 (note however that unlike the methods in U.S. Patent Application No. 2012/0322058, the present methods do not include a step of introducing an agent that cleaves the sample nucleic acids). Droplet digital PCR (ddPCR) divides PCR samples into partitions (e.g., water-in-oil droplets). See, e.g., Hindson et al., 2011, Anal. Chem. 83:8604-8610; Pinheiro et al., 2012, Anal. Chem. 84:1003-1011. The droplets support PCR amplification of the template molecules, if present, and use reagents that are capable of specifically generating a signal from target amplicons, i.e., amplicons from the target sequences. For example, a primer pair that specifically amplifies the first target sequence and a separate primer pair that specifically amplifies the second target sequence linked to the first target sequence is present or delivered to each partition. Additional primers can be included if more target or control sequences are to be generated. Exemplary reagents can also include probes that generate a fluorescent signal upon binding the relevant target sequence. Exemplary probes include but are not limited to Taqman probes, Scorpion probes and molecular beacons. In some embodiments, probes for each different target produce a different wavelength signal allowing for each to be separately detected. Following PCR, signal from each droplet is read to determine the number of positive droplets for each target amplified in the original sample (including partitions having multiple different targets as well as portions only having single or no target signal).

Methods and compositions for partitioning are described, for example, in published patent applications WO 2010/036,352, US 2010/0173,394, US 2011/0092,373, and US 2011/0092,376. The plurality of partitions can be in a plurality of emulsion droplets, or a plurality of nanowells, microwells, etc.

In some embodiments, one or more reagents are added during droplet formation or to the droplets after the droplets are formed. Methods and compositions for delivering reagents to one or more partitions include microfluidic methods as known in the art; droplet or microcapsule combining, coalescing, fusing, bursting, or degrading (e.g., as described in U.S. 2015/0027,892; US 2014/0227,684; WO 2012/149,042; and WO 2014/028,537); droplet injection methods (e.g., as described in WO 2010/151,776); and combinations thereof.

As described herein, the partitions can be picowells, nanowells, or microwells. The partitions can be pico-, nano-, or micro-reaction chambers, such as pico, nano, or microcapsules. The partitions can be pico-, nano-, or micro-channels. The partitions can be droplets, e.g., emulsion droplets.

In some embodiments, the partitions are droplets. In some embodiments, a droplet comprises an emulsion composition, i.e., a mixture of immiscible fluids (e.g., water and oil). In some embodiments, a droplet is an aqueous droplet that is surrounded by an immiscible carrier fluid (e.g., oil). In some embodiments, a droplet is an oil droplet that is surrounded by an immiscible carrier fluid (e.g., an aqueous solution). In some embodiments, the droplets described herein are relatively stable and have minimal coalescence between two or more droplets. In some embodiments, less than 0.0001%, 0.0005%, 0.001%, 0.005%, 0.01%, 0.05%, 0.1%, 0.5%, 1%, 2%, 3%, 4%, 5%, 6%, 7%, 8%, 9%, or 10% of droplets generated from a sample coalesce with other droplets. The emulsions can also have limited flocculation, a process by which the dispersed phase comes out of suspension in flakes. In some cases, such stability or minimal coalescence is maintained for up to 4, 6, 8, 10, 12, 24, or 48 hours or more (e.g., at room temperature, or at about 0, 2, 4, 6, 8, 10, or 12° C.). In some embodiments, the droplet is formed by flowing an oil phase through an aqueous sample or reagents.

The oil phase can comprise a fluorinated base oil which can additionally be stabilized by combination with a fluorinated surfactant such as a perfluorinated polyether. In some embodiments, the base oil comprises one or more of a HFE 7500, FC-40, FC-43, FC-70, or another common fluorinated oil. In some embodiments, the oil phase comprises an anionic fluorosurfactant. In some embodiments, the anionic fluorosurfactant is Ammonium Krytox (Krytox-AS), the ammonium salt of Krytox FSH, or a morpholino derivative of Krytox FSH. Krytox-AS can be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of Krytox-AS is about 1.8%. In some embodiments, the concentration of Krytox-AS is about 1.62%. Morpholino derivative of Krytox FSH can be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of morpholino derivative of Krytox FSH is about 1.8%. In some embodiments, the concentration of morpholino derivative of Krytox FSH is about 1.62%.

In some embodiments, the oil phase further comprises an additive for tuning the oil properties, such as vapor pressure, viscosity, or surface tension. Non-limiting examples include perfluorooctanol and 1H,1H,2H,2H-Perfluorodecanol. In some embodiments, 1H,1H,2H,2H-Perfluorodecanol is added to a concentration of about 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.25%, 1.50%, 1.75%, 2.0%, 2.25%, 2.5%, 2.75%, or 3.0% (w/w). In some embodiments, 1H,1H,2H,2H-Perfluorodecanol is added to a concentration of about 0.18% (w/w).

In some embodiments, the sample is partitioned into, or into at least, 500 partitions, 1000 partitions, 2000 partitions, 3000 partitions, 4000 partitions, 5000 partitions, 6000 partitions, 7000 partitions, 8000 partitions, 10,000 partitions, 15,000 partitions, 20,000 partitions, 30,000 partitions, 40,000 partitions, 50,000 partitions, 60,000 partitions, 70,000 partitions, 80,000 partitions, 90,000 partitions, 100,000 partitions, 200,000 partitions, 300,000 partitions, 400,000 partitions, 500,000 partitions, 600,000 partitions, 700,000 partitions, 800,000 partitions, 900,000 partitions, 1,000,000 partitions, 2,000,000 partitions, 3,000,000 partitions, 4,000,000 partitions, 5,000,000 partitions, 10,000,000 partitions, 20,000,000 partitions, 30,000,000 partitions, 40,000,000 partitions, 50,000,000 partitions, 60,000,000 partitions, 70,000,000 partitions, 80,000,000 partitions, 90,000,000 partitions, 100,000,000 partitions, 150,000,000 partitions, or 200,000,000 partitions.

In some embodiments, the droplets that are generated are substantially uniform in shape and/or size. For example, in some embodiments, the droplets are substantially uniform in average diameter. In some embodiments, the droplets that are generated have an average diameter of about 0.001 microns, about 0.005 microns, about 0.01 microns, about 0.05 microns, about 0.1 microns, about 0.5 microns, about 1 microns, about 5 microns, about 10 microns, about 20 microns, about 30 microns, about 40 microns, about 50 microns, about 60 microns, about 70 microns, about 80 microns, about 90 microns, about 100 microns, about 150 microns, about 200 microns, about 300 microns, about 400 microns, about 500 microns, about 600 microns, about 700 microns, about 800 microns, about 900 microns, or about 1000 microns. In some embodiments, the droplets that are generated have an average diameter of less than about 1000 microns, less than about 900 microns, less than about 800 microns, less than about 700 microns, less than about 600 microns, less than about 500 microns, less than about 400 microns, less than about 300 microns, less than about 200 microns, less than about 100 microns, less than about 50 microns, or less than about 25 microns. In some embodiments, the droplets that are generated are non-uniform in shape and/or size.

In some embodiments, the droplets that are generated are substantially uniform in volume. For example, the standard deviation of droplet volume can be less than about 1 picoliter, 5 picoliters, 10 picoliters, 100 picoliters, 1 nL, or less than about 10 nL. In some cases, the standard deviation of droplet volume can be less than about 10-25% of the average droplet volume. In some embodiments, the droplets that are generated have an average volume of about 0.001 nL, about 0.005 nL, about 0.01 nL, about 0.02 nL, about 0.03 nL, about 0.04 nL, about 0.05 nL, about 0.06 nL, about 0.07 nL, about 0.08 nL, about 0.09 nL, about 0.1 nL, about 0.2 nL, about 0.3 nL, about 0.4 nL, about 0.5 nL, about 0.6 nL, about 0.7 nL, about 0.8 nL, about 0.9 nL, about 1 nL, about 1.5 nL, about 2 nL, about 2.5 nL, about 3 nL, about 3.5 nL, about 4 nL, about 4.5 nL, about 5 nL, about 5.5 nL, about 6 nL, about 6.5 nL, about 7 nL, about 7.5 nL, about 8 nL, about 8.5 nL, about 9 nL, about 9.5 nL, about 10 nL, about 11 nL, about 12 nL, about 13 nL, about 14 nL, about 15 nL, about 16 nL, about 17 nL, about 18 nL, about 19 nL, about 20 nL, about 25 nL, about 30 nL, about 35 nL, about 40 nL, about 45 nL, or about 50 nL.

The methods involve determining (a) the number of first partitions that contain a first nucleic acid linked to the second nucleic acid, (b) the number of first partitions that contain the first nucleic acid without the second nucleic acid and (c) the number of first partitions that contain the second nucleic acid without the first nucleic acid. The number of (a) can be determined, for example, as the number of partitions that display signal from probes for both nucleic acid sequences. Optionally, overabundance of partitions with both probe signals in a partition compared to what is expected from random dispersion of the two probes' signals can indicate that the sample contained polynucleotides that have at least two targets nucleic acid sequences linked. In other words, one can assess whether, and to what extent, the number of partitions with a particular combination of targets is in statistical excess compared to what would be expected if the targets were randomly distributed in the partitions. The extent of overabundance of such partitions can be used to estimate the number of linked targets.

In some embodiments, the method further comprises enumerating the number of partitions comprising a reference nucleic acid sequence, which can be used to normalize the number of first nucleic acid, second nucleic acid and any further nucleic acid sequences assayed. In some embodiments, the number of copies of the first nucleic acid and second nucleic acid is normalized to the number of occurrences of the reference sequence. In some embodiments, the sample is from a human and the reference nucleic acid sequence is at least a portion of the RPP30 gene. For example, the first nucleic acid and second nucleic acid can be normalized to RPP30, e.g., calculated per a volume (e.g., 20 microliter) of the reaction, to determine a serial score for copy numbers using RPP30 control gene as the normalizer for each day of specimen collection (e.g., Score=[(N1+N2)/2]/RPP30, where N1 and N2 represent the N1 and N2 of SARS-CoV-2, but can be any first and second target nucleic acid as described herein).

The methods described herein can be performed on one sample or multiple samples (e.g., from the same subject over time, for example, once a day or one every other day) allowing one to characterize the infectious agent in the subject by assessing the relative viability or degradation of the infectious agent. In some embodiments, a single sample is obtained from the subject and the linkage of the two or more target nucleic acid sequences is quantified as detailed above, for example the number of partitions containing linked sequences and the number of unlinked sequences is determined. In this case, the resulting number of partitions for linked or unlinked sequences or both or a ratio of linked to unlinked, or ratio of linked or unlinked to total (linked plus unlinked), each of which can be normalized as described herein, can be compared to one or more threshold value to categorize the results. Thus, for example, a threshold value can be determined for separating contagious individuals from non-contagious individuals based on the absolute amount of linked to unlinked sequences or the ratio of linked to unlinked sequences or ratio of linked or unlinked to total (linked plus unlinked) and this threshold value can then be compared to data from an infected individual to characterize the infectious agent and thus predict whether the individual is in a contagious stage of disease. As an example, a relatively high number of linked target sequences can indicate that the infectious agent is viable and for example an individual carrying it is contagious, or at least more contagious than if the number was lower. In some embodiments, an increased occurrence of unlinked target sequences (e.g., where partitions contain one but not the second, typically-linked target sequence) can indicate the infectious agent has been degraded in the subject and thus the subject may be less contagious. The precise threshold value can be selected based on the sensitivity and specificity desired by the user and can be determined for example, based on measuring and averaging results from a series of infected individuals as they pass through different stages of the infectious disease.

In some embodiments, two or more (e.g., 2, 3, 4, 5, or more) samples can be obtained from the subject over time. In these embodiments, the number of linked or unlinked or both or the ratio of linked to unlinked or ratio of linked or unlinked to total positive (linked plus unlinked) [percent linkage] target sequences can be compared to one or more threshold value as discussed above, or one or more of the number of linked or unlinked or both or the ratio of linked to unlinked target sequences or ratio of linked or unlinked to total positive (linked plus unlinked) from one sample can be compared to a second (or more) sample. This latter option can be useful, for example, for characterizing the infectious agent in the subject over time, e.g., thereby monitoring the course of infection, when a subject is likely contagious or not, or for example how well the subject is responding to a treatment.

In some embodiments, the subject is provided with a treatment or course of care determined by how the infectious agent is categorized by the methods described herein. For example, if the subject is determined to carry viable infectious agent (e.g., above a threshold) the subject can be treated with antibiotics, anti-viral or other agents that will ameliorate the infection or symptoms caused by the infection.

A system for performing the methods disclosed herein is also provided. The system may comprise a droplet generator configured to form droplets of an aqueous phase including nucleic acid. The system also may comprise a thermocycler and a detector configured to collect amplification data (e.g., signal at different wavelength to detect different amplified nucleic acid sequences) from individual droplets. The system further may comprise a processor. The processor may be configured to the determine the number of positive partitions for the various target nucleic acids, as well as for normalizing the data and optionally for comparing the data to a threshold value or data from different samples that can be stored in memory. In some embodiments, the system comprises Bio-Rad QX200 (or QXDx AutoDG or QX ONE) Droplet Digital PCR system (Hercules, Calif.).

In one aspect, a computer program product is provided comprising a non-transitory machine readable medium storing program code that, when executed by one or more processors of a computer system, causes the computer system to implement at least one step of a method as described herein, for example comparing the number of partitions containing linked or unlinked target nucleic acid sequences from a first sample to a threshold value or comparing the number(s) to such numbers from a second sample.

### Example

We have investigated the utility of droplet digital PCR (ddPCR) to determine absolute viral copies in quantifying the viral load as well as examine the quality of the viral nucleic acids in pre-symptomatic, symptomatic, asymptomatic, and convalescent individuals. We show multiple examples of serial testing for presence of two proximal amplification regions within the Nucleocapsid (N) gene in the SARS-CoV-2 genome, N1 and N2. The data provided herein demonstrates that absolute quantification using ddPCR allows for accurate measurement of viral copy numbers and further, the levels of amplicon linkage tracks with the progression of infection.

In pre-symptomatic individuals, we observed highly linked N1 and N2 PCR gene products that maintained this state as the viral copies rise with the onset of symptoms. In individuals with mild or no reported symptoms the linkage and rise in viral load are similarly observed. The degree of linkage rapidly declines as the individual recovers. The viral genomes presumably degrade and are cleared as the individual enters convalescence. Results in our study demonstrate that although there is some turn-over at all stages of infection, the SARS-CoV-2 virus is intact and actively replicating earlier during infection, and vastly out-paces the rate of degradation at peak viral loads. This replication rate is rapid in the first 2-7 days of the infection and is reflected in a high degree of intact virus (high linkage). By contract, the viral load and linkage between N1 and N2 rapidly diminish, indicating genome degradation as the infection wanes and the individual enters convalescence.

This is the first report detailing the observation of serial changes in gene linkage in the context of quantifiable viral copy numbers in SARS-CoV-2 infected individuals. We expect these methods to have broad utility in other diseases, especially those caused by human pathogens.

### Materials and Methods:

The FDA EUA method for the SARS-CoV-2 ddPCR test was developed by Bio-Rad and commercialized by Biodesix for use in Biodesix's centralized CAP/CLIA/CLEP certified laboratory. Briefly, nasal swab specimens are collected into a transport media and transported by courier to the laboratory for testing by ddPCR. Viral specimens are inactivated, RNA is extracted and then subjected to droplet generation, thermo cycling and analysis on a partition (droplet) fluorescence reader (e.g., QX-200; Bio-Rad Inc.).

The SARS-CoV-2 assay included a single tube, triplex assay that is based on the current, validated CDC assay. Specifically, the assay is capable of detecting viral Targets (N1—Nucleocapsid 1 and N2—Nucleocapsid 2) as well as a control target (RPP30—human gene encoding RNase P). The primary specimen type is a nasal swab specimen and as used by Biodesix has been validated for use with a variety of transport Media including but not limited to the PrimeStore® Molecular Transport Medium, Amies Medium, Norgen Total Nucleic Acid Preservation Tubes, Saline, as well as various Universal Transport Medium (UTM)/Viral Transport Medium (VTM) types including Hardy Diagnostics™ VTM, RMBIO® VTM, MicroTest™ M4RT™, iClean® VTM, MedSchenker™ Smart Transport Medium, and AccuViral UTM.

Three controls are run with every batch of clinical samples. A human cell line (A549; ATCC) is used for RNA extraction monitoring; a commercially sourced standard consisting of synthetic Nucleocapsid RNA transcripts in genomic DNA background (Exact, Bio-Rad SKU COVID19) was used for a positive RT-ddPCR control; and a no template negative control (nuclease-free water) is used to monitor the RT-PCR reaction for potential contamination.

RNA was extracted using Quick-RNA Viral 96 kit from Zymo Research (cat #R1040, R1041). An extraction control sample was processed with each batch. 300 μL of the transport media was mixed with 300 μL inactivation solution (DNA/RNA Shield, Zymo Research). 200 μL of the sample/shield mixture was combined with 400 μL Viral RNA buffer and applied to a 96-well spin column plate. The plate was centrifuged for 5 minutes at 2200×g. The columns were washed twice with 500 μL Viral wash buffer and once with 100% ethanol; after application of each wash the plate was centrifuged for 5 minutes at 2200×g and the flowthrough was discarded. The plate was then spun for an additional 2 minutes at 2200×g to dry the columns. 75 μL nuclease-free water was applied to each column, and the plate was centrifuged for 5 minutes at 2200×g to elute the RNA. The RNA was held on ice until use in ddPCR followed by storage in ultra-low freezer.

For single column extractions (column from Zymo Research, D4014), all volumes were the same, but centrifugation speeds and times differed. Columns were spun at 10,000×g for all steps: 2 minutes for binding, 30 seconds for each of the washes, and 2 minutes for the drying spin. Purified RNA was eluted into 1.5 mL tubes and held on ice until use in ddPCR followed by storage in ultra-low freezer.

The reaction mix was 5.5 μL RNA and 16 μL PCR master mix (Table 1); 20 μL of this was used to generate droplets on a QX200 Droplet Generator (Bio-Rad). A positive and negative control was processed with each batch. The droplets were transferred to a 96 well PCR plate and run on a combined RT-ddPCR thermocycling program (Table 2). After thermocycling, the plate was transferred to a QX200 droplet reader (Bio-Rad). The results from the reader were analyzed to determine copy numbers of N1, N2, and RPP30 detected in each 20 μL PCR. Labels for 2D droplet clusters were generated based on thresholds for each target.

Mean percent linkage of N1 and N2 was calculated as follows:

$$Mean ( ( 100 \times ( N 1 linkage value ) / ( N 1 copies / µL ) ) , ( 100 \times ( N 2 linkage value ) \text{} / ( N 2 copies / µL ) ) ) ; “ linkage value ” = linked molecules / µl .$$

TABLE 1. PCR Master Mix for detection of COVID-19 and RPP30.

TABLE 2. PCR Program for COVID-19 RT-ddPCR reaction.

Table 3. Serial Detection of SARS-CoV-2 N1 and N2 copies, and human control gene RNase P (RPP30), in one representative donor. Nasal swab specimens were analyzed prior to molecular positivity (day 0), through the pre-symptomatic, asymptomatic, symptomatic, asymptomatic (recovery), and convalescence (molecular negative) stages of infection, using a SARS CoV-2 ddPCR test. A. shows the total copy numbers of the viral N genes and the human control gene RPP30; B shows the % linkage of the N1 and N2 genes over the course of the infection.

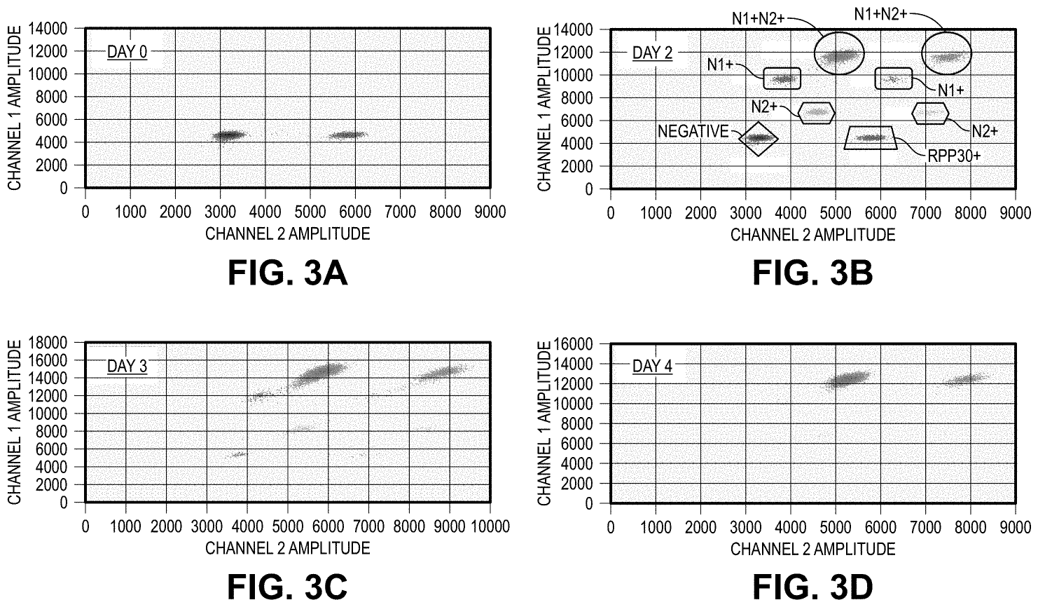

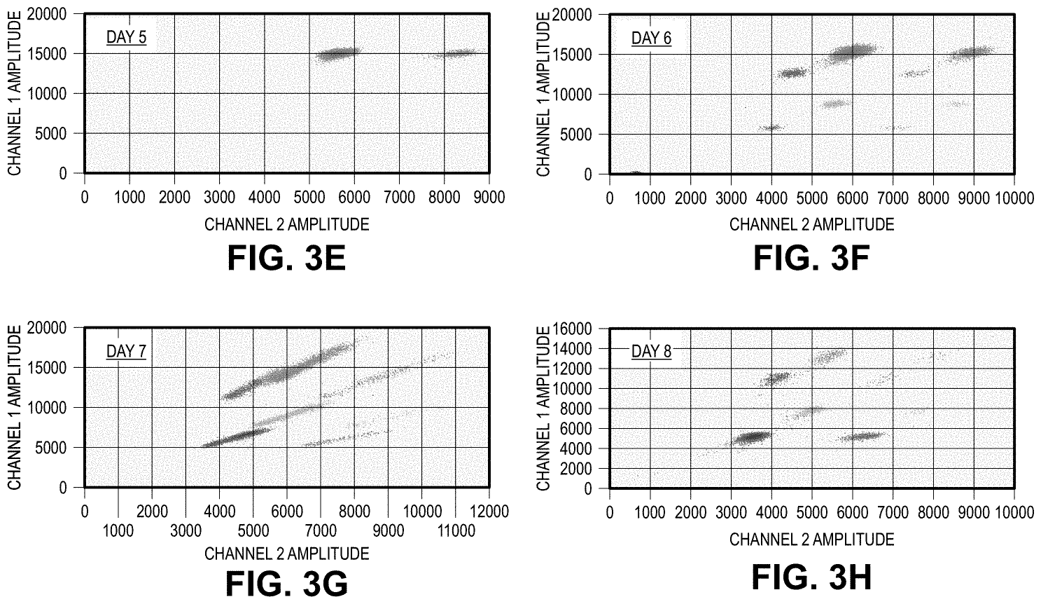

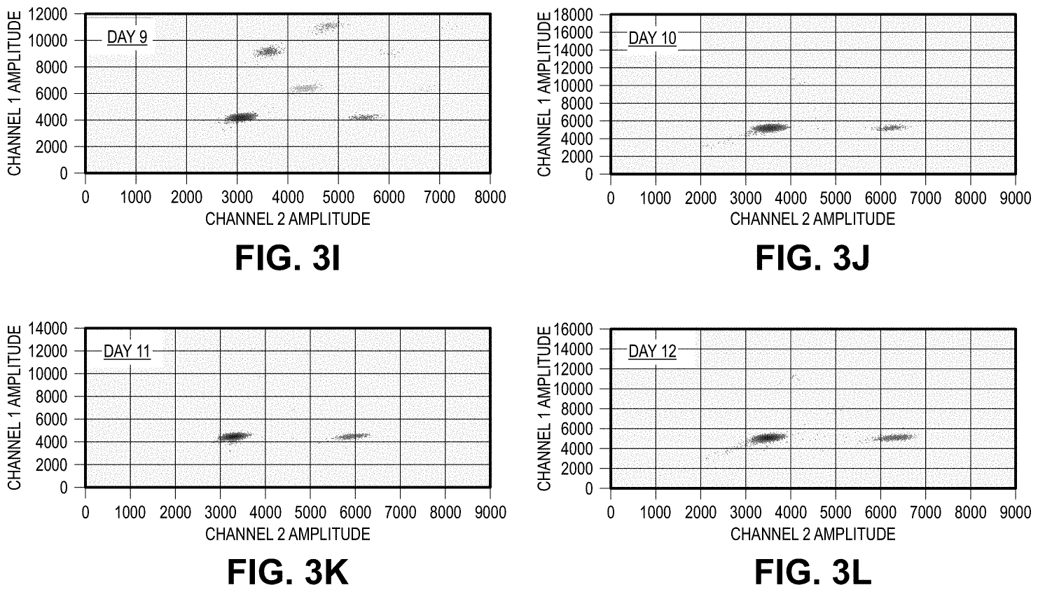

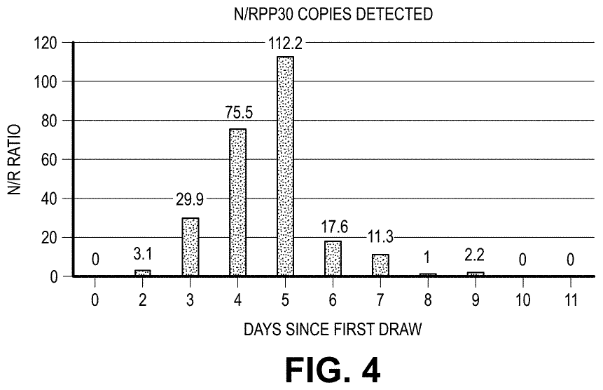

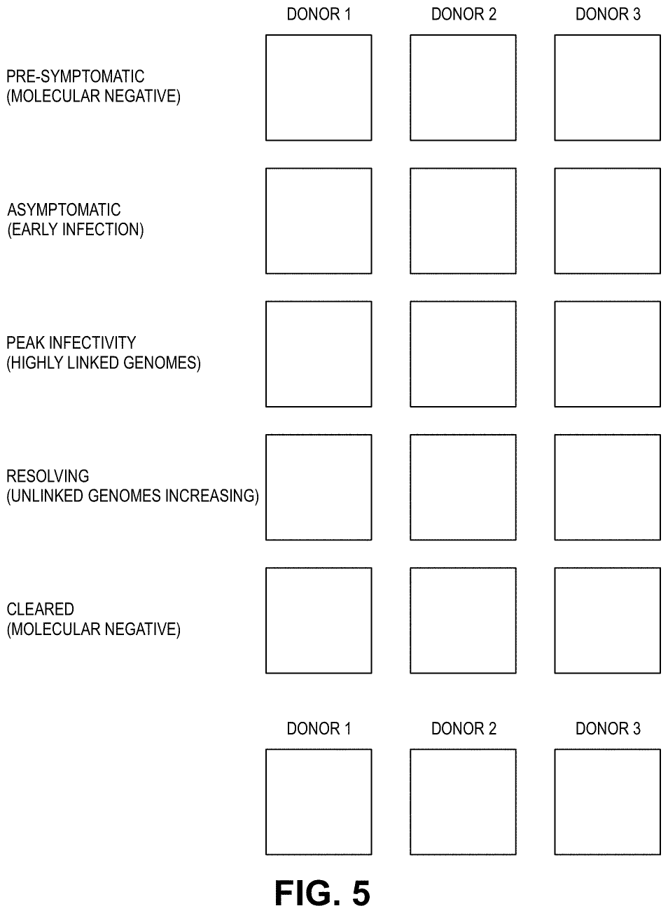

These data show the increase in the viral copy numbers from pre-infection, through the asymptomatic, and symptomatic stages. Concomitant with this, the observation is that the percentage linkage increases for N1 and N2, peaks at 100% and then declines rapidly as the virus is cleared in this representative donor series. These data are shown visually in the 2D plots from QuantaSoft in FIG. 3a-c, and in the calculated viral load (FIG. 4). The rise in viral load (Table 4) and genome quality (FIG. 3b-e) likely represent an increased likelihood of viral transmissibility and infectivity.

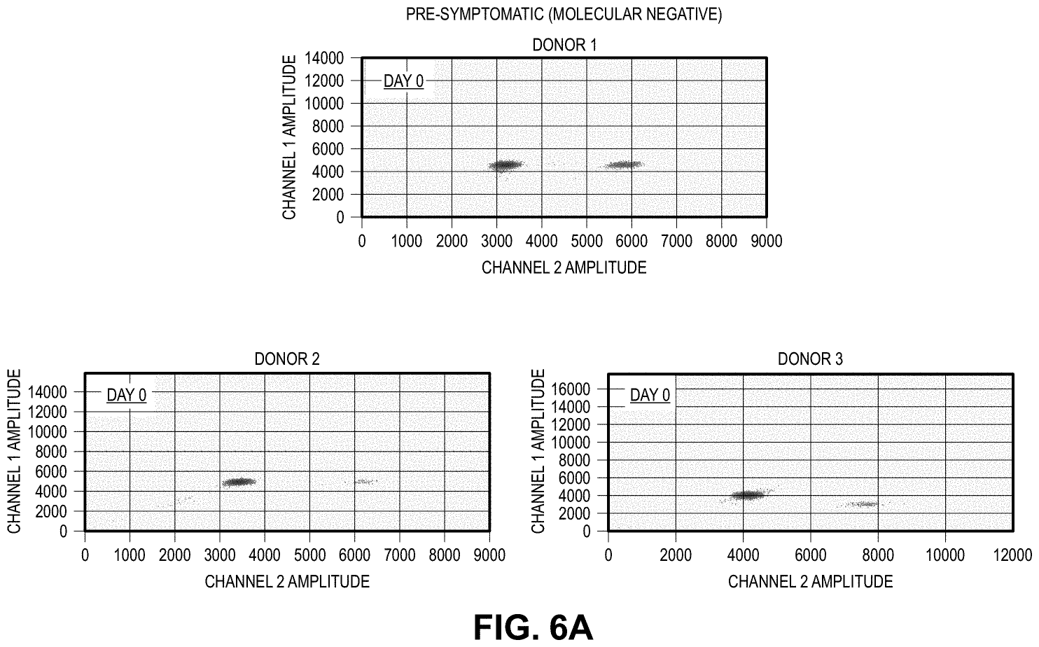

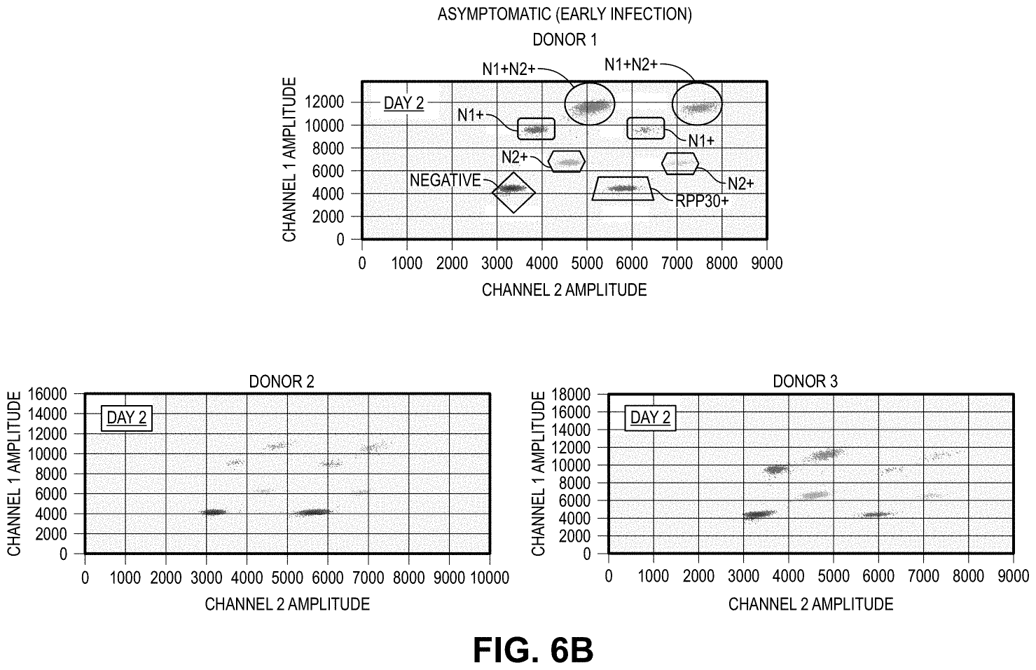

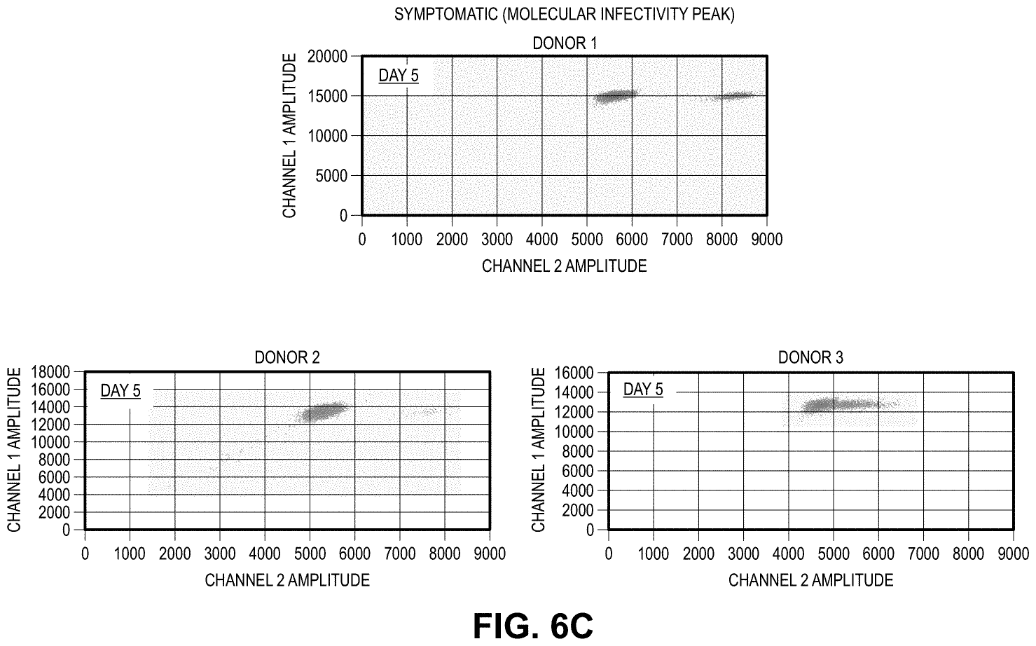

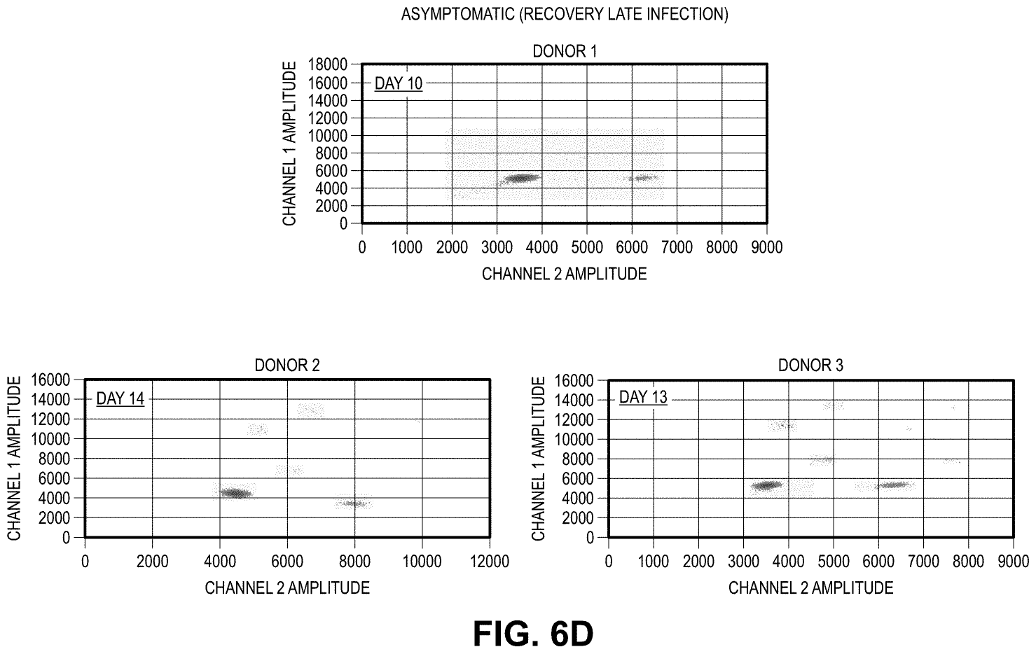

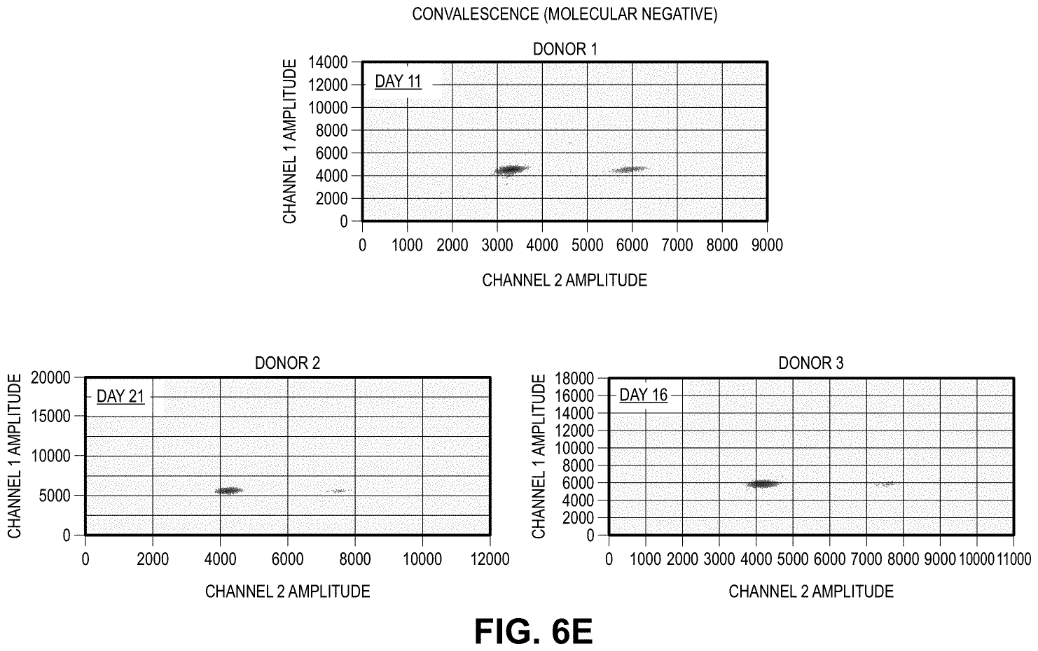

Symptomatic individuals are known to be highly transmissible and examples of clinical cases are shown in FIG. 11. Similar molecular linkage clusters are observed whether these clinical cases were diagnosed as symptomatic or asymptomatic, indicating that either case is likely infectious. This report additionally shows a molecular mechanism that clearly demonstrates that other stages of diagnosis are molecularly similar to symptomatic individuals, and replicating, intact genomes, are detected in individuals who are pre-symptomatic (FIG. 3b; day 2) and are asymptomatic (FIG. 6b, c). These individuals are as likely to be transmitting the virus by virtue of having similar viral loads and high quantity of intact genomes especially early on in infection. We show additional examples of linked (infectious) and unlinked (degraded genomes) using serial ddPCR results. Results for donors 2 and 3 (FIG. 6 and Table 4) showed similar results as observed in the full serial series for donor 1 (FIG. 3, FIG. 6, Table 3, 4). Viral loads can be seen increasing during the active infection phase and then decreasing rapidly. Linkage profiles for all donors were high during active infection and low following peak viral load (FIG. 3, 6 and Tables 3, 4). These data collectively demonstrate a common and measurable phenotype that can differentiate very early, early, peak, and late infection with a combination of absolute copy numbers. viral load, cluster distribution and linkage values generated using ddPCR.

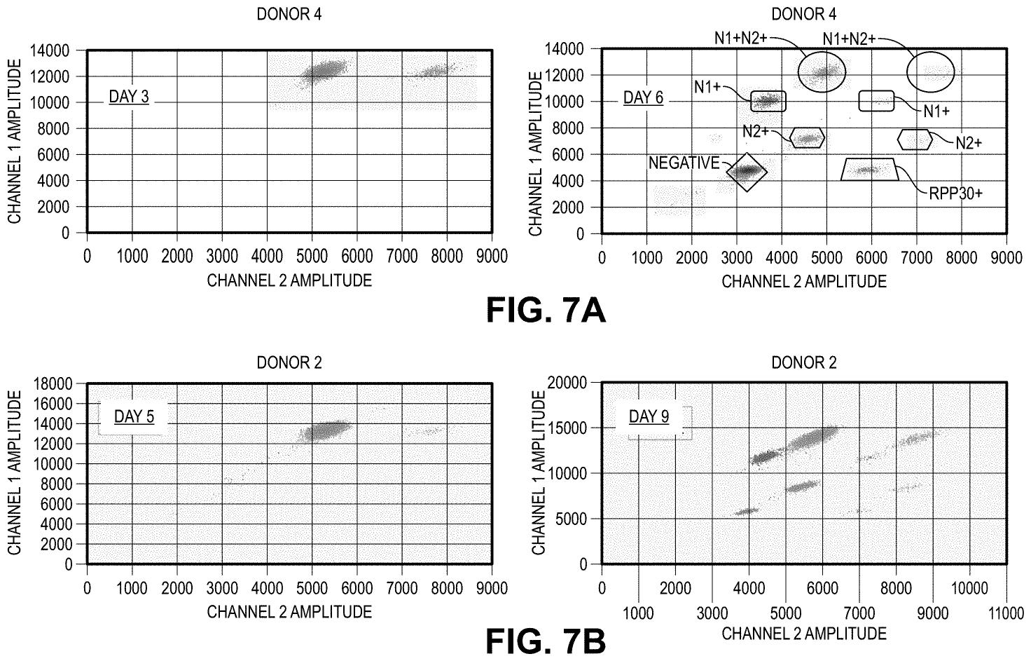

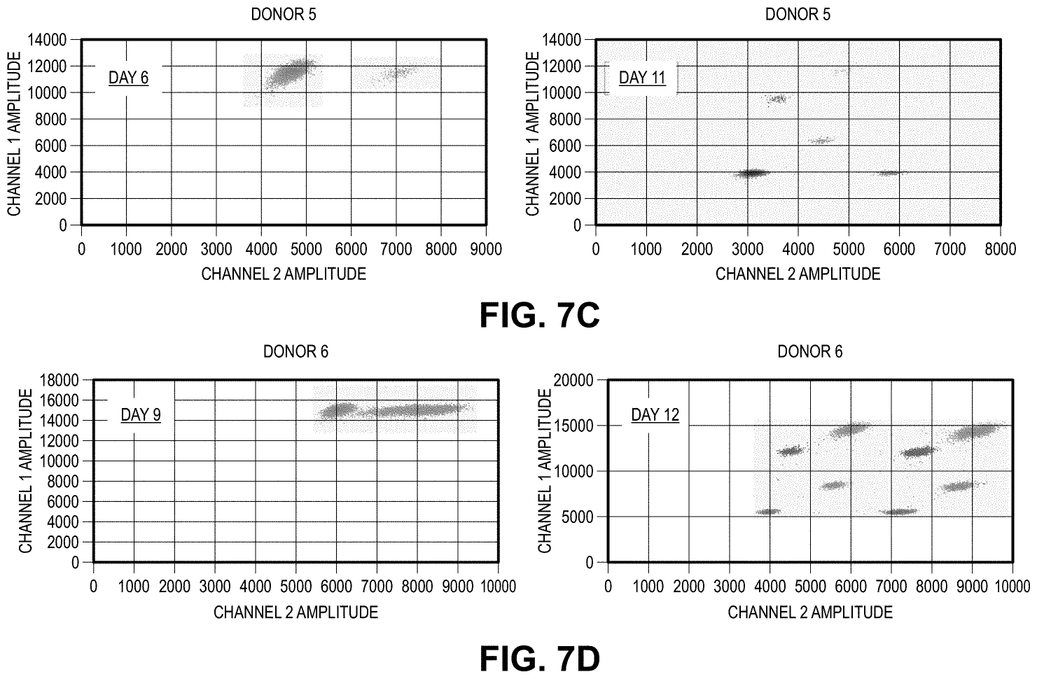

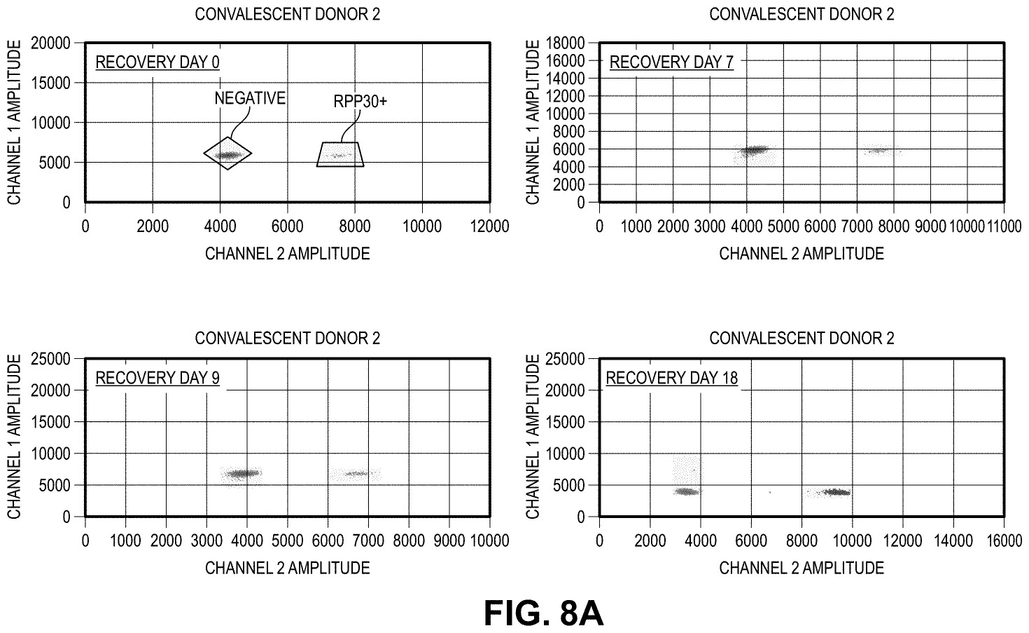

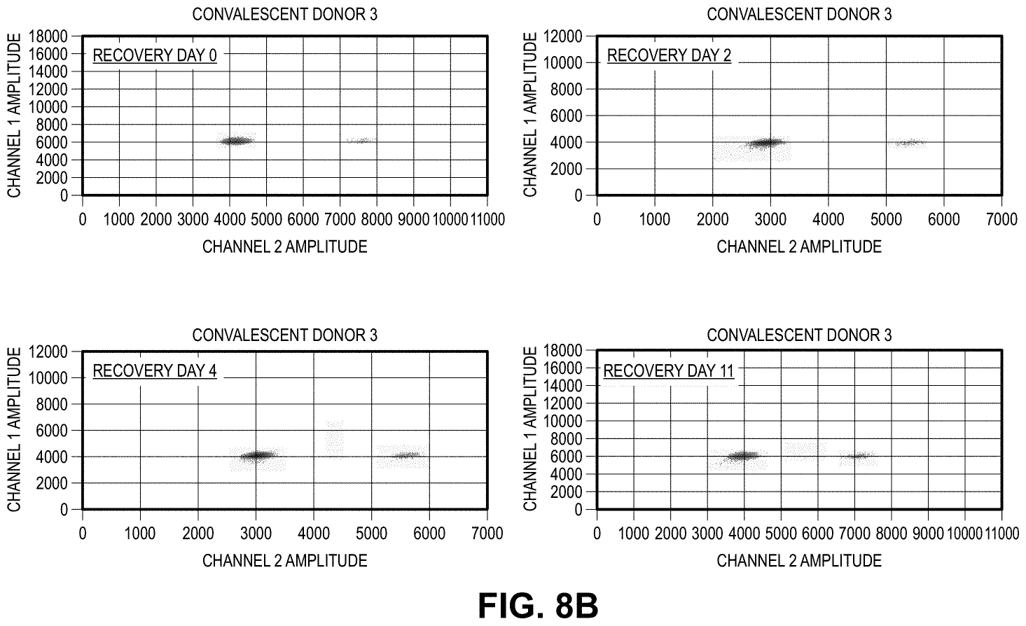

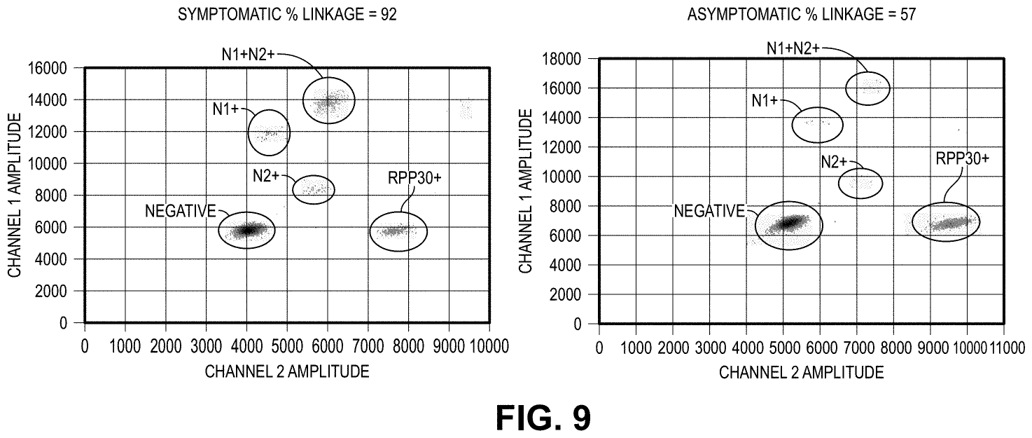

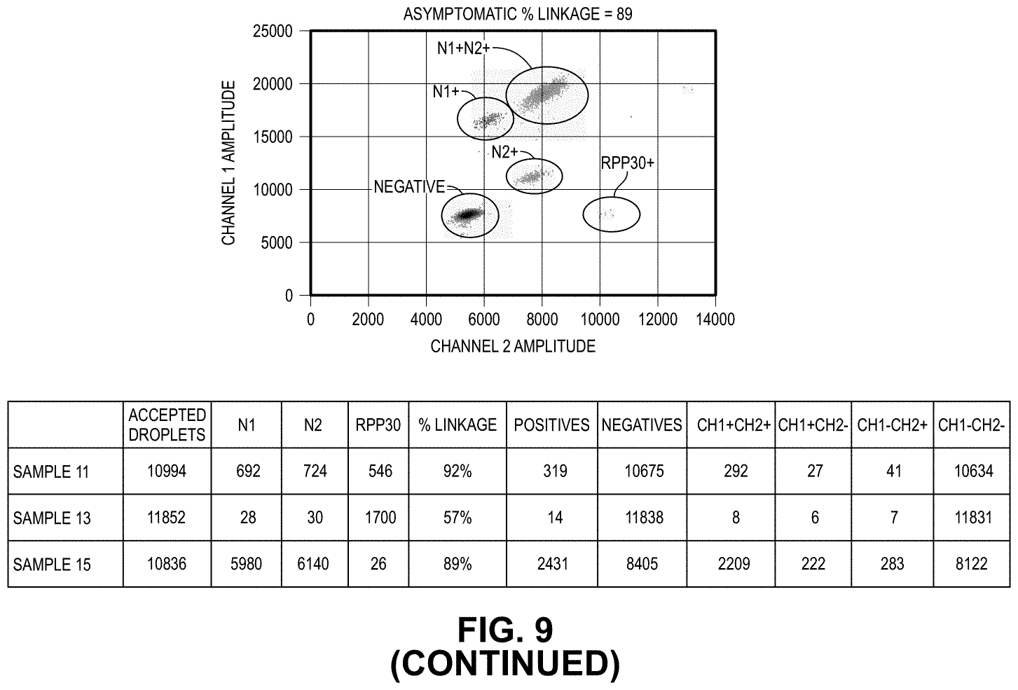

We further describe the clustering phenotype (FIG. 7, and viral load and linkage calculation (Table 5) for asymptomatic, mildly symptomatic, and severe symptoms (required hospitalization and/or oxygen). The kinetics of viral load and linkage were similar for patients with symptoms or not (see also FIG. 7 and highlight the need for a molecular analysis such as described in this study to identify patients that are likely to be infections by the virtue of having linked, replication competent genomes. Studies of convalescent donors were also conducted in an attempt to identify potential infectious genomes late in infection and during convalescence (FIG. 8). Here we show an example of three individual cases at multiple time points following recent SARS-CoV-2 infection. Even at these early time points post-recovery we have not yet observed viral loads nor appreciable linkage. The effect of viral replication and linkage is confined to the infection cycle when the virus is most transmissible and further indicates that recovered individuals are less or unlikely to be infections for the same strain of the virus that they were previously infected with.

### REFERENCES

1. Wang, C., et al., A novel coronavirus outbreak of global health concern. Lancet, 2020. 395(10223): p. 470-473.
2. Walsh, K. A., et al., SARS-CoV-2 detection, viral load and infectivity over the course of an infection. J Infect, 2020. 81(3): p. 357-371.
3. La Scola, B., et al., Viral RNA load as determined by cell culture as a management tool for discharge of SARS-CoV-2 patients from infectious disease wards. Eur J Clin Microbiol Infect Dis, 2020. 39(6): p. 1059-1061.
4. Curtis J. Mello, N. K., Heather de Rivera, Steven A. McCarroll, Absolute quantification and degradation evaluation of SARS-CoV-2 RNA by droplet digital PCR. medRxiv preprint, 2020.

The above examples are provided to illustrate the invention but not to limit its scope. Other variants of the invention will be readily apparent to one of ordinary skill in the art and are encompassed by the appended claims. All publications, databases, internet sources, patents, patent applications, and accession numbers cited herein are hereby incorporated by reference in their entireties for all purposes.

What is claimed is:

1. A method of characterizing an infectious agent in a subject, the method comprising,
providing a first sample from the subject comprising infectious agent nucleic acids;
partitioning the first sample into a plurality of first partitions;
detecting in the first partitions the presence or absence of a first infectious agent nucleic acid and a second infectious agent nucleic acid, wherein the first infectious agent nucleic acid and the second infectious agent nucleic acid are covalently linked in a viable infectious agent nucleic acid;
determining (a) the number of first partitions that contain the first infectious agent nucleic acid linked to the second infectious agent nucleic acid and (b) the number of first partitions that contain the first infectious agent nucleic acid without the second infectious agent nucleic acid or (c) the number of first partitions that contain the second infectious agent nucleic acid without the first infectious agent nucleic acid; and
characterizing the infectious agent in the subject based on the determining of (a) and (b) or (a) and (c).

2. The method of claim 1, wherein the determining comprises determining (b) and (c) and the characterizing is based on the determining of (a) and (b) and (c).

3. The method of claim 1, wherein the characterizing comprises comparing (a), (b), (c) or a combination thereof to one or more threshold value.

4. The method of claim 1, further comprising,
providing a second sample from the subject comprising infectious agent nucleic acids, wherein the second sample was obtained from the subject at a later time point than the first sample;
partitioning the second sample into a plurality of second partitions;
detecting in the second partitions the presence or absence of a first infectious agent nucleic acid and a second infectious agent nucleic acid;
determining (a′) the number of second partitions that contain the first infectious agent nucleic acid linked to the second infectious agent nucleic acid, (b′) the number of second partitions that contain the first infectious agent nucleic acid without the second infectious agent nucleic acid and (c′) the number of second partitions that contain the second infectious agent nucleic acid without the first infectious agent nucleic acid;
wherein the characterizing comprises comparing (a) to (a′), (b) to (b′), (c) to (c′) or a combination thereof.

5. The method of claim 4, wherein the second sample was obtained from the subject at least 24 hours (e.g., 1-10, 1-5, 1-3, 1-2 days) after the first sample was obtained.

6. The method of claim 1, further comprising detecting in the partitions a control nucleic acid and wherein the determining comprises normalizing:
(a) the number of first partitions that contain the infectious agent nucleic acid linked to the second infectious agent nucleic acid, and
(b) the number of first partitions that contain the first infectious agent nucleic acid without the second infectious agent nucleic acid, and/or
(c) the number of first partitions that contain the second infectious agent nucleic acid without the first infectious agent nucleic acid,
to the number of partitions containing the control nucleic acid.

7. The method of claim 1, wherein the characterizing comprises categorizing the infectious agent as viable or degraded.

8. The method of claim 1, wherein the infectious agent is a virus.

9. The method of claim 8, wherein the infectious agent is a virus selected from the group consisting of SARS-CoV-2, influenza, and respiratory syncytial virus (RSV).

10. The method of claim 8, wherein the infectious agent is SARS-CoV-2.

11. The method of claim 10, wherein the first infectious agent nucleic acid comprises at least a detectable portion of nucleocapsid (N) gene N1 and the second infectious agent nucleic acid comprises at least a detectable portion of N gene N2.

12. The method of claim 1, wherein the infectious agent is a bacterium or a mycoplasma.

13. The method of claim 1, wherein the first infectious agent nucleic acid and the second infectious agent nucleic acid are separated by 100-10,000 nucleotides from each other in the viable infectious agent nucleic acid.

14. The method of claim 1, wherein the subject is a human.

15. The method of claim 1, wherein the partitions are droplets in an emulsion or microwells or nanowells.

16. A method of characterizing an infectious agent in a subject, the method comprising,
providing a first sample from the subject comprising infectious agent nucleic acids;
determining (a) an amount of first infection agent nucleic acid linked to the second infection agent nucleic acid, (b) an amount of first infectious agent nucleic acid unlinked to second infectious agent nucleic acid and (c) optionally an amount of second infectious agent nucleic acid unlinked to first infectious agent nucleic acid; and
characterizing the infectious agent in the subject based on the determining of (a), (b) and optionally (c).
